(19) United States
(12) Patent Application Publication
(10) Pub. No.: US 2019/0021430 A1
(43) Pub. Date: Jan. 24, 2019

(54) Hat with flex to store or use eyeshield

(71) Applicant: Steven Michael Elliott, Gig Harbor, WA (US)

(72) Inventor: Steven Michael Elliott, Gig Harbor, WA (US)

(21) Appl. No.: 15/655,861

(22) Filed: Jul. 20, 2017

(51) Int. Cl. A42B 1/24; A42B 1/06; A61F 9/04

(52) U.S. Cl. CPC A42B 1/247; A61F 9/045; A42B 1/062

(57) ABSTRACT

An eyeshield made of flexible transparent plastic is attached to the underside of a curved brim of an article of headwear by a connection which will let the eyeshield flex from an open, viewable position to a closed hidden position. By utilizing the formed curvature of the brim, the eyeshield can be flexed into a concaved closed position against the underside of the brim. By manually pulling the rear, unattached edge of the eyeshield down from the stored position, the eyeshield will flex into a convex shape and lock into an open, viewable position perpendicular to the brim of the article of headwear.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

### 1.) Field of Invention

This invention relates to attaching an eyeshield to the brim of an article of headwear such as a hat.

### 2.) Background Description of Art

There has been many methods of attaching an eyeshield to a hat and most have created a highly visible mechanical means of attaching this eyeshield. Some methods have the ability to flip the eyeshield from a horizontal position to a vertical position but the hinge has always been a highly visible component of the hat. One method solved this by attaching the eyeshield to the inside headband of a hat but this method requires removal of the hat in order to deploy the eyeshield plus this method also places the eyeshield closely in front of the eyes making it unusable for eyeglass wearers.

This new invention creates a very durable, inexpensive, and fashionable means of attaching an eyeshield to the brim of a hat. It overcomes all of the problems listed above plus those associated with the wearing and storing of eyeshields for those who wear corrective eyeglasses.

### BRIEF SUMMARY OF THE INVENTION

The main purpose of this invention is to create a very durable, fashionable, and cost effective method for wearing an eyeshield to help protect eyeglasses and eyes from rain, sun, and particulates. This invention will also be able to deploy and use or put away and store an eyeshield at a moments notice. When stored, the eyeshield will be protected and virtually unnoticable by the wearer and any observers.

### BRIEF DESCRIPTION OF THE SEVERAL VIEWS OF THE DRAWING

FIG. 1 shows in a perspective view, a baseball hat with the eyeshield attached to the brim and in an open, viewable position perpendicular to the brim.

FIG. 2 shows in a perspective view similar to FIG. 1 but at a lower angle showing the underside of the brim of a hat with the eyeshield in its open position.

FIG. 3 shows in a perspective view, a baseball hat with the eyeshield attached to the brim and in its closed position.

FIG. 4 shows a front view of a hat with the eyeshield in its open and viewable position.

FIG. 5 shows a front view of a hat with the eyeshield in its closed and stored position.

FIG. 6 shows the underside of the brim of a hat with the eyeshield attached by the flexible hinge and in a closed position.

FIG. 7 shows the eyeshield in a flat view.

### DETAILED DESCRIPTION OF THE INVENTION

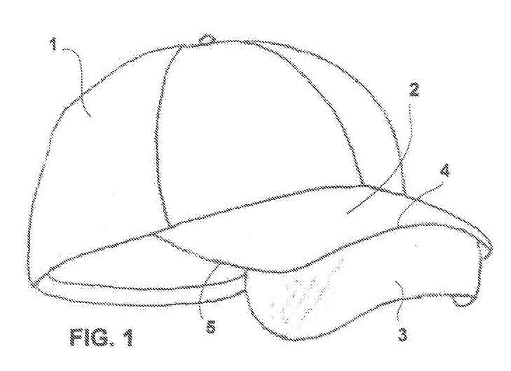

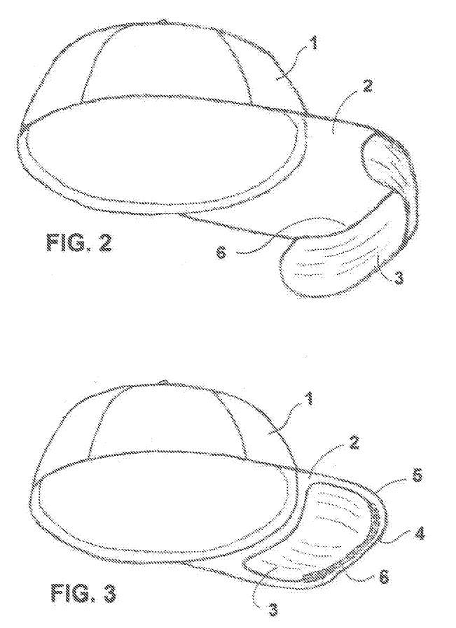

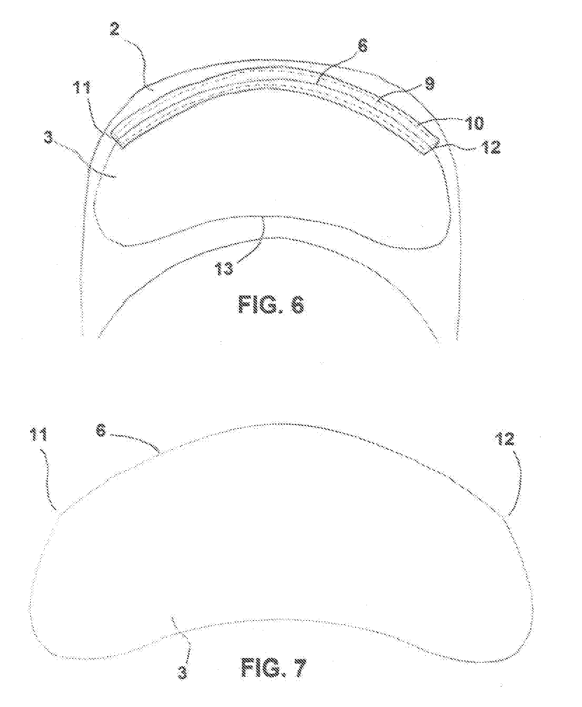

This invention uses any style of hat 1 which has a curved brim 2 as a component of the article of headwear needed for this eyeshield 3 to function. An example of this style of hat is shown in FIG. 1. The eyeshield 3 is attached to the underside and towards the front leading edge of the brim 2. The eyeshield 3 is flexible enough to be able to curve around and follow the front leading edge of the brim 4 and continue to follow part way around the side edge of the brim 5. In FIG. 2 the top edge of the eyeshield 6 is shown attached to the underside of the brim 2. This is accomplished by a flexible hinge 9 made out of but not limited to a braided elastic as shown in FIG. 6.

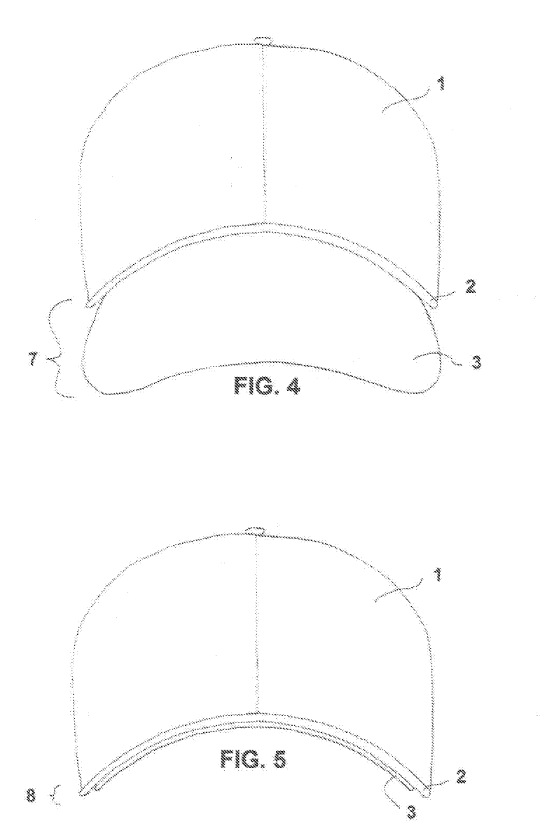

When the eyeshield 3 is in its open and viewable position as shown in FIG. 4, the eyeshield will be facing up and down and perpendicular to the brim 7. When the eyeshield 3 is in its closed and stored position, as shown in FIG. 5, the eyeshield will be flexed against and parallel to the underside of the brim 8, making the eyeshield virtually unnoticable by both wearer and observer.

The forward side of the flexible hinge 9 is attached to the underside of the brim 2 either by sewing with thread 10 or glueing as shown in FIG. 6. This attachment of the flexible hinge 9 to the underside of the brim 2 will follow very closely the shape of the eyeshield. The other side of the flexible hinge is attached to the eyeshield 3 following the top leading edge 6 of the eyeshield. This attachment is made either by sewing with thread 10 or glueing. The flexible hinge 9 attaches the eyeshield 3 to the underside of the brim 2 from anchor point 11 on one side of the eyeshield, all along the top edge of the eyeshield 6, to the anchor point 12 on the other side of the eyeshield. The eyeshield is first flexed into a convex curved position following the curvature manufactured into the brim 2 as shown in FIG. 5 and then the hinge with eyeshield is attached to the underside of the brim at anchor points 11 and 12, shown in FIG. 6. This causes the eyeshield to stay in a flexed convex position paralleling the curvature of the hats brim while the eyeshield is in its closed, stored position, as in FIG. 5. The remaining hinge section between anchor point 11 and 12 can now be attached to the underside of the brim as the eyeshield is now being forced into the curvature of the brim. The brim 2 will have a curved radius of between 7 cm-10 cm or 2.75″-4.0″ and will be made with a curved plastic insert inside a cloth sleeve common to most baseball hat styles as in FIG. 1.

The eyeshield 3 is to be a shape similar as shown in FIG. 7. The top edge of the eyeshield 6 is designed to follow the arch curvature of the underside of the brim 2 while the eyeshield 3 is in its flexed concave, open, and viewable position as in FIG. 2. This shape is of an oblong curved design which allows the eyeshield 3 to be able to pivit from a parallel position in relationship to the brim 2 as in FIG. 3 to a perpendicular position in relationship to the brim while being affixed to the brim with the flexible hinge 9 as shown in FIG. 2. The eyeshield 3 can be flexed from its closed convex position to its open concave position by grabbing the lower edge of the eyeshield 13 shown in FIG. 6 and pull down. As the eyeshield 3 flexes to its concave, open position, the top edge of the eyeshield 6 which is attached to the underside of the brim 2 by the flexible hinge 9 will pivot from a parallel position as shown in FIG. 5 to a perpendicular position, as shown in FIG. 4. This causes the eyeshield now to be in the open, viewable position.

The eyeshield 3 is made of a flexible transparent plastic such as but not limited to polycarbonate. It will have a thickness of between 0.50 mm-0.75 mm or about 0.020″-0.030″. It can either be clear as to be used for rainwater protection for eyeglass wearers or tinted for sunlight protection for all wearers. The size of the eyeshield 3 will be smaller in both length and width of the brim 2 in order to store out of the way under the brim 2 as shown in FIG. 3.

1. An eyeshield attached by a hinge to the underside and towards the leading edge of the curved brim of an article of headwear in which the curved shape of this brim is utilized to flex the eyeshield into two usable positions which are a stored concave position underneath and parallel to this brim or into a viewable convex position perpendicular and towards the leading edge of this brim.

2. The curved shape of the brim of this article of headwear in claim 1 is from left to right of the brim with the center of the brim being the highest part of the arch and the left and right side edges being the lower part of the arch and equal in length to the center of the brim.

3. The curved shape of the brim in claim 1 will have a radius of between 7 cm-10 cm or 2.75″-4.0″.

4. The curved shape of the brim in claim 1 is created by using an insert such as but not limited to a piece of plastic formed into this 7 cm-10 cm radius and sewn inside a sleeve made of fabric and is common in the making of a brim for a baseball cap.

5. The hinge in claim 1 allows the eyeshield to rotate from a stored concave position parallel to the underside of the brim to a viewable convex position perpendicular to the brim while the hinge is attached onto the eyeshield and brim in an arch shape following the shape of the top edge of the eyeshield.

6. One side of the hinge in claim 5 is attached to the top edge of the eyeshield and follows the oblong shape of the eyeshield from a distinct point on the left side of the eyeshield to a distinct point on the right side of the eyeshield.

7. One side of the hinge in claim 5 is attached to the curved brim of the article of headwear towards the front leading edge of the brim.

8. The hinge in claim 6 will be attached to the curved brim and to the edge of the eyeshield by a method of sewing with thread or gluing.

9. The hinge in claim 5 is made of a flexible material which allows the hinge to stretch in length for making the arch following the top edge of the eyeshield.

10. The eyeshield in claim 1 is designed in shape as to where the top edge of the eyeshield will follow the oblong shape of the underside curvature of the brim when the eyeshield is in its open convex position perpendicular to the brim.

11. The eyeshield in claim 10 is made of a flexible plastic such as but not limited to polycarbonate.

12. The eyeshield in claim 10 will be between 0.50 mm-0.75 mm or 0.020″-0.030″ in thickness.

13. The eyeshield in claim 10 is of consistent thickness.

14. The eyeshield in claim 10 will be either clear for rainwater protection or tinted for sun protection.

15. The eyeshield in claim 10 has two distinct points on the top edge of the eyeshield where the eyeshield will no longer follow the contour of the underside of the brim when the eyeshield is in its open position.

16. The two distinct points on the eyeshield in claim 15 are where the hinge starts and ends the attachment of the hinge to the eyeshield.

17. The two distinct points on the eyeshield in claim 15 are where the hinge starts and ends the attachment of the hinge to the underside of the brim.

18. The eyeshield in claim 10 will be equal to or less than the width of the brim from left to right so that the eyeshield will be unnoticeable in its closed and stored position parallel to the inside of the brim.

19. The eyeshield in claim 10 will be equal to or less than the length of the brim from the front where the eyeshield is attached to the underside of the brim to the rear of the brim where the brim attaches to the crown or body of the article of headwear.
